(19) United States
(12) Patent Application Publication
(10) Pub. No.: US 2023/0066394 A1
(43) Pub. Date: Mar. 2, 2023

(54) VIRTUAL AND DIGITAL RESEARCH MODEL AND RELATED METHODS FOR IMPROVING ANIMAL HEALTH AND PERFORMANCE OUTCOMES

(71) Applicant: Premex, Inc., Durham, NC (US)

(72) Inventors: Alejandro MESA, Durham, NC (US); Jorge ESTRADA, Durham, NC (US); Ruben VARGAS, Durham, NC (US)

(21) Appl. No.: 17/800,183

(22) PCT Filed: Feb. 17, 2021
(86) PCT No.: PCT/US2021/018290

§ 371 Date: Aug. 16, 2022

Related U.S. Application Data
(60) Provisional application No. 62/977,434, filed on Feb. 17, 2020.

(51) Int. Cl. A01K 29/00; G06N 5/04

(52) U.S. Cl. CPC A01K 29/005; G06N 5/041

(57) ABSTRACT

A method and system for conducting virtual and digital research is provided. The method and systems seek to improve livestock health and outcomes.

### CROSS-REFERENCE TO RELATED APPLICATIONS

The present application claims priority to U.S. Application Ser. No. 62/977,434 filed Feb. 17, 2020, the contents of which are incorporated herein by reference.

### BACKGROUND OF THE DISCLOSURE

The traditional approach to conduct research in livestock animals aims to understand one or two variables in an animal's environment at the same time, while standardizing other variables/conditions to get responses based on a hypothesis. Conditions are highly variable within a production system as well as differences between regions or countries. Furthermore, the animal protein production worldwide is based on biological systems that are highly complex. Livestock animals are raised under many variable conditions such that understanding the interaction between various factors, and how these factors affect the welfare and performance of the animals has been of great challenge for the livestock industry.

### SUMMARY OF THE DISCLOSURE

A method of conducting digital research to improve livestock health outcomes is provided. The method includes the steps of forming a hypothesis regarding what changes may be made to one or more variables related to livestock nutrition, environment, health status, or animal performance to improve livestock health outcomes for a particular livestock producer; performing a cluster analysis of historical data related to the one or more variables to determine one or more smart farms for real-time livestock data collection; obtaining real-time livestock data from the selected one or more smart farms; analyzing the real-time livestock data to test the hypothesis; and generating a customized research report that includes results of the analysis of the real-time livestock data including one or more recommendations to improve livestock health outcome. According to one embodiment, the one or more recommendations may relate to suggested changes to one or more variables to improve livestock health outcomes. According to one embodiment, the real-time livestock data is obtained from a plurality of sensors located on or around a plurality of livestock at the smart farm. According to one embodiment, the plurality of sensors transmits a particular code associated with an individual livestock animal or individual smart farm. According to one embodiment, the step of analyzing the livestock data is accomplished by one or more statistical models. According to one embodiment, the cluster analysis utilizes historic data obtained from a plurality of smart farms located across one or more global regions. According to one embodiment, the method further includes the step of transmitting the research report wirelessly to the one or more smart farms; and confirming or denying acceptance of the research report by the one or more smart farms.

A method for conducting virtual research to predict the outcome of a selected variable of interest and improve livestock health outcomes is provided. The method includes the steps of:

(a) obtaining livestock data from a plurality of livestock producers, the data related to one or more variables pertaining to livestock nutrition, environment, health or performance;

(b) transmitting the livestock data to a cloud computing environment;

(c) analyzing the livestock data in the cloud computing environment with one or more analysis models; and

(d) generating a customized report that includes the results of the analysis, the results including an estimate or prediction of an expected improvement in health outcome by changing one or more variables. According to one embodiment, the selected variable of interest may be one or more of a microbiome, feeding method, feeding schedule, medical history, breed, breeding status, age, body condition, genetics, gender, environmental temperature, humidity, water pH, water temperature, water quality, ammonia levels in environment, air quality, barn flooring quality/type, feeder type, feed quality, feed size, nutritional levels and requirements, disease, medication, medication delivery method, distance of livestock from other barns and/or packing plants, growth performance, and carcass characteristics. According to one embodiment, the livestock data includes data obtained from the Internet of Animals. According to one embodiment, the step of analyzing the livestock data includes applying artificial intelligence to the livestock data in a plurality of data processing modules within a cloud computing environment in which the plurality of data processing modules are executed in conjunction with at least one processor, the data processing modules and artificial intelligence configured to continuously evaluate data from a plurality of livestock producers to estimate or predict an expected improvement in health outcome by changing one or more variables. According to one embodiment, the method further includes the step of transmitting the report to an end user, wherein the end user may accept or deny the report. According to one embodiment, upon acceptance of the report, the method includes the steps of: implementing the change to one or more variables in a field trial; analyzing the data produced from the field trial; and retraining the one or more of the analysis models. According to one embodiment, upon denial of the report, the method includes the step of: repeating the steps (a)-(d) until one or more variables dependent on improvement through several simulations is fulfilled or until a maximum number of iterations have been raised.

A system for digital research is provided. The digital research system includes at least one database; at least one server; at least one livestock producer interface including a data entry system, the livestock producer interface in wireless communication with a gateway and the at least one server; at least one livestock sensor, the livestock sensor coupled to a gateway that is in wireless communication with the at least one database and the at least one server; an artificial intelligence component configured to analyze one or more variables and recommend a change to one or more variables; and a memory and processor in wireless communication with the at least one database, at least one server, livestock producer interface, at least one livestock sensor and artificial intelligence component. According to one embodiment, the digital research system further includes at least one livestock scale or camera, the livestock scale or camera coupled to a gateway that is in wireless communication with the at least one database, at least one server, memory and processor. According to one embodiment, the at least one database, at least one server, memory, artificial intelligence component, and processor are configured to process one or more of producer input data, sensor data, and any normalized data to recommend a change to one or more variables.

A system for virtual research is provided. The virtual research system includes a cloud computing environment including a statistical model component configured to analyze stored data and estimate or predict an expected improvement in health outcome by changing one or more variables; and one or more databases comprising stored data, the one or more databases in wireless communication with the cloud computing environment. According to one embodiment, the one or more databases includes access to the Internet of Animals. According to one embodiment, the statistical model component includes artificial intelligence. According to one embodiment, the virtual research system further includes a dashboard, the dashboard in wireless communication with the cloud computing environment.

These and other features and advantages of the present disclosure will be further understood and appreciated by those skilled in the art by reference to the following specification, claims and appended drawings.

### BRIEF DESCRIPTION OF THE DRAWINGS

FIG. 1 is a flowchart of steps for a method of conducting virtual research according to one embodiment.

FIG. 2 is a flowchart of steps for a method of conducting digital research according to one embodiment.

### DETAILED DESCRIPTION OF THE DISCLOSURE

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements.

The embodiments described below may assume various alternative orientations and step sequences, except where expressly specified to the contrary. Specific devices and any related processes illustrated in the attached drawings, and described in the following specification are simply exemplary embodiments of the concepts defined in the appended claims. Hence, specific dimensions and other physical characteristics relating to the embodiments disclosed herein are not to be considered as limiting, unless the claims expressly state otherwise.

As used in the specification, and in the appended claims, the singular forms “a”, “an”, “the”, include plural referents unless the context clearly dictates otherwise.

As used in the specification, and in the appended claims, the term “Internet of Animals” refers to stored or historical data related to animal behavioral patterns and movement. Such data from the Internet of Animals may be historical data that is stored in an accessible central database and gathered from animals from one or more regions around the world.

As used in the specification, and in the appended claims, the term “livestock” refers to one or more animals kept or raised in an agricultural or farm setting for pleasure or profit.

As used in the specification, and in the appended claims, the term “medicament” refers to a substance used for medical treatment.

As used in the specification, and in the appended claims, the term “server” refers to a computer in a network utilized to process and provide data to other computers or components.

As used in the specification, the term “smart farm” refers to a concept of farming management that utilizes various technological devices, such as sensors, to gather data to aid in the improvement of animal health and performance outcomes.

The traditional approach of livestock research requires many trials with the same objective that need to be carried out in each system (sometimes several times within a company) to understand the effects of a specific variable associated with livestock health/performance outcomes. The methods as provided herein may simulate and predict multiple scenarios under different conditions while at the same time allowing for the prediction of the likeliest outcome when a variable is modified within a system on the health and performance of livestock. The methods as provided herein may supplement or replace commercial research currently conducted in livestock production systems.

At least two methods are proposed for investigation on commercial farms. The first method creates a predictive model that explains a high percentage of the variance of the system. Thus, the size of the experiments on commercial farms is significantly reduced. Once accounted for the predicted variance, the results can be analyzed using statistic techniques. A second method performs virtual simulations focused on treatments with the highest expected improvement or best health outcome. The best simulation results may be chosen for implementation in the field or on a commercial livestock operation. The results from the virtual simulations may be taken back to retrain the artificial intelligence and perform new simulations.

### Virtual Research Method

According to one embodiment, the virtual research method seeks to build simulations that allocate confidence on a combination of independent variables previously observed in historical data. The method explores, via simulation, combinations of independent variables which maximize improvement in livestock health and performance. By not utilizing farm resources to gather data, expenses for research is greatly reduced. The virtual research method allows for simulations to be run with combinations of variables that might not have been implemented by a livestock producer.

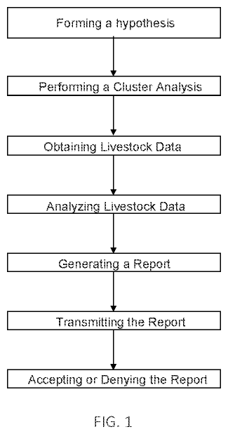

A method for conducting virtual research to predict the outcome of a selected variable of interest and improve livestock health outcomes is provided (see FIG. 1; (100)). The method includes the steps of:

(a) obtaining livestock data from a plurality of livestock producers, the data related to one or more variables pertaining to livestock nutrition, environment, health or performance (102);

(b) transmitting the livestock data to a cloud computing environment (104);

(c) analyzing the livestock data in the cloud computing environment with one or more analysis models (106); and

(d) generating a customized report that includes the results of the analysis, the results including an estimate or prediction of an expected improvement in health outcome by changing one or more variables (108).

According to one embodiment, the method includes the step of obtaining data from the Internet of Animals to reveal data related to behavioral patterns and movement. Such data from the Internet of Animals may be historical data stored in a central database.

According to one embodiment, the livestock data from a plurality of livestock producers may pertain to one or more variables related to livestock nutrition, environment, health status, or animal performance. According to one embodiment, the data pertaining to such variables may be historical data stored in a central database. According to one embodiment, the variable data obtained may pertain to microbiome, feeding method, feeding schedule, medical history, breed, breeding status, age, body condition, genetics, gender, environmental temperature, humidity, water pH, water temperature, water quality, ammonia levels in environment, air quality, barn flooring quality, feeder type, feed quality, feed size, presence of toxins in feed (e.g., mycotoxins), fatty acid composition of fats and oils in feed, feed processing techniques (e.g., pelleting, expellers, particle size), disease, medication, medication delivery method, distance of livestock from other barns for packing plants, growth performance (e.g., growth rate, feed conversion, mortality), and carcass characteristics (e.g., lean, backfat, prime cuts yield), transportation of time from hatchery to farm (for chickens, turkeys, etc.), transportation time from farm to a processing plant, age of mother at time of birth (or egg laying), and difference of weight between biological mother and father.

The method of conducting virtual research further includes the step of analyzing the livestock data to produce a simulation that predicts what combinations of independent variables will maximize an expected livestock health improvement. According to one embodiment, the step of analyzing the livestock data is accomplished via processing in the cloud configuration provided herein.

According to one embodiment, the step of analyzing the livestock data includes correlation, normalization, aggregation and modeling of the data to predict a variable change or expected output with the goal of improving animal health. According to one embodiment, the step of analyzing the livestock data is accomplished through the application of various models such as lambda function, artificial intelligence, machine learning or application of artificial intelligence. According to one embodiment, the step of analyzing the livestock data is accomplished through the application of a surrogate model. The surrogate model can be gaussian processes, deep neural networks, decision trees, or tree of parzen estimation.

According to one embodiment, the virtual research method includes the step of building an artificial intelligence supervised learning model. According to one embodiment, the artificial intelligence supervised learning model seeks to explain any variability in performance. These models include any model provided herein such as, for example, ensemble learning, deep learning, time series modeling, or Bayesian statistics.

According to one embodiment, the step of analyzing the livestock data includes utilizing a plurality of data processing modules within a computing environment in which the plurality of data processing modules are executed in conjunction with at least one processor, the data processing modules and artificial intelligence configured to continuously evaluate the data to simulate and ultimately predict an expected output or a variable change with the goal to improve animal health.

According to one embodiment, the method further includes the step of generating a customized research report that includes a summary of the simulation and general results of the analysis. According to one embodiment, the customized report includes an estimate or prediction of an expected improvement in outcome by changing a one or more previously unexplored combinations of independent variables. According to one embodiment, the customized report includes a recommendation for changing one or more variables to improve animal health. According to one embodiment, the report may include a prediction or suggested recommendation that includes a modification to one of the variables provided herein. By improving animal health, livestock owners may increase profits.

According to one embodiment, the method optionally includes the step of transmitting the report to an end user. According to one embodiment, the report may be viewed electronically on a smart device or customized interface.

According to one embodiment, the method optionally includes the step of the end user accepting or denying the report.

According to one embodiment, the method of conducting virtual research includes the step of repeating the steps provided herein to continue to make new simulations until a variable dependent on improvement through several simulations is fulfilled or until a maximum number of iterations have been raised.

According to one embodiment, the method of conducting virtual research includes the step of implementing a recommendation (i.e., best simulation) through controlled trials in the field (such as by a livestock owner). According to one embodiment, the method of conducting virtual research includes the step of analyzing the data produced as a result of implementation of the recommendation by a livestock producer. According to one embodiment, the method of conducting virtual research includes the step of retraining one or more of the analysis models provided herein. According to one embodiment, the method of conducting virtual research includes the step of retraining the artificial intelligence model. According to one embodiment, the method of conducting virtual research includes the step of retraining the artificial intelligence model via semi-supervised learning, transfer learning, or from scratch.

### Virtual Research System

According to one aspect, a system for virtual research is provided. According to one embodiment, the virtual research system includes a cloud computing environment including a statistical model component configured to analyze stored data and estimate or predict an expected improvement in health outcome by changing one or more variables; and one or more databases comprising stored data, the one or more databases in wireless communication with the cloud computing environment. According to one embodiment, the one or more databases includes access to the Internet of Animals. According to one embodiment, the statistical model component includes artificial intelligence. According to one embodiment, the virtual research system further includes a dashboard, the dashboard in wireless communication with the cloud computing environment.

According to one embodiment, the virtual research system includes at least one server. The at least one server may be located in a cloud configuration. According to one embodiment, the system includes at least one computer coupled to the at least one server. The at least one computer may be located in a cloud configuration and coupled or in communication with the at least one server. The computer includes at least one processor and memory for analyzing the data as provided herein.

According to one embodiment, the system for virtual research further includes server access to one or more databases or other servers having access to the Internet of Animals as well as data related to one or more variables provided herein. Such servers and databases may be in wireless communication with one or more servers or databases in a cloud computing environment. The cloud computing environment may include at least one server, at least one data storage device (such as a database) and at least one processor. The cloud computing environment may include or otherwise store one or more of the statistical models described herein. The cloud computing environment may include or otherwise store one or more of the statistical model components including artificial intelligence or machine learning component configured to analyze one or more variables and predict an effect of the selected variable.

According to one embodiment, the system further includes at least one database coupled to or in wireless communication with a gateway that is in wireless communication with the at least one server, memory, artificial intelligence component, and processor. According to one embodiment, a database is provided that stores and maintains various standardized stored data with respect to each of the Internet of Animals, health profiles of particular livestock types, the genetic information of particular livestock types (herd or individual livestock) and data related to one or more variables provided herein. According to one embodiment, the database maintains one or more licenses to access one or more stored data services which aid in and provide stored data related to disease diagnosis, prescriptions medicines, health assessment data and genetic data. According to one embodiment, the stored data is received, standardized or normalized and stored for later use. According to one embodiment, all standard data is cleaned, aggregated and stored before further processing.

According to one embodiment, the data and reports as provided herein is entered into or received into a cloud pipeline system. According to one embodiment, the cloud pipeline system includes at least one server, processor and memory to receive, analyze, process, and store the various data as provided herein. According to one embodiment, the cloud pipeline system includes more than one or a plurality (e.g., cluster) of servers receive, process and store the various data as provided herein. According to one embodiment, the cloud pipeline system includes at least one computer to receive, analyze, process, detect anomalies, and store the various data as provided herein. According to one embodiment, at least one server is configured to receive, process and store the various data as provided herein. The number of servers, computers or a combination thereof is scalable and varies depending on data size.

According to one embodiment, a report is generated and provided by the methods and systems provided herein. The report may include various identifying information such as a client or user identification number and time stamp. According to one embodiment, the report is provided wirelessly via a specific dashboard for viewing. The dashboard is in wireless communication with the cloud computing environment. According to one embodiment, the report is provided wirelessly via email or stored on a central database for central access.

According to one embodiment, data and reports, such as sensor data as provided herein, may be managed and stored in a NoSQL (“non SQL” or “non-relational”) database such as that provided by Apache Cassandra. According to one embodiment, the NoSQL database provides a mechanism for storage and retrieval of data and reports as provided herein that is modeled in means other than the tabular relations used in relational databases.

### Digital Research Method

The present disclosure also provides a method of conducting digital research to improve livestock health outcomes. The digital research methods provided herein seek to utilize analyzing models to differentiate between top variables influencing the performance variance and the other variables. The digital research method predictive model seeks to explain a high percentage of the variance of the system, therefore, the size of the experiments on livestock producer farms is significantly reduced.

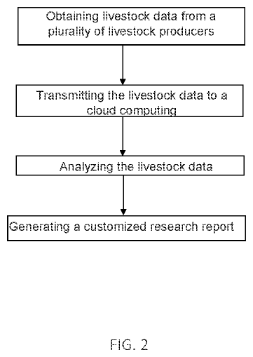

As illustrated in FIG. 2, the method includes the steps of forming a hypothesis (202) regarding what changes may be made to one or more variables related to livestock nutrition, environment, health status, or animal performance to improve livestock health outcomes for a particular livestock producer; performing a cluster analysis (204) of historical data related to the one or more variables to determine one or more smart farms for real-time livestock data collection; obtaining real-time livestock data (206) from the selected one or more smart farms; analyzing the real-time livestock data (208) to test the hypothesis; and generating a customized research report (210) that includes the results of the analysis. The digital research method may further include the steps of transmitting the research report (212) wirelessly to the one or more smart farms; and confirming or denying acceptance of the research report (214) by the one or more smart farms.

The method includes the step of forming a hypothesis regarding what changes to various variables provided herein may be changed to improve livestock health and related outcomes. The hypothesis may be formed based on a known variable with the intention of predicting the likeliest outcome when a variable is modified within a system on the health, welfare and performance of livestock. The hypothesis may be based on a report with recommendations from simulations produced in the virtual research model provided herein.

The variables that may be changed include those provided herein such as those that pertain to one or more variables related to livestock nutrition, environment, health status, or animal performance. According to one embodiment, the data variables may pertain to one or more health or environmental variables including, but not limited to, microbiome, feeding method, feeding schedule, medical history, breed, breeding status, age, body condition, genetics, gender, environmental temperature, humidity, water pH, water temperature, water quality, ammonia levels in environment, air quality, barn flooring quality, feeder type, feed quality, feed size, presence of toxins in feed (e.g., mycotoxins), fatty acid composition of fats and oils in feed, feed processing techniques (e.g., pelleting, expellers, particle size), disease, medication, medication delivery method, distance of livestock from other barns for packing plants, growth performance (e.g., growth rate, feed conversion, mortality), and carcass characteristics (e.g., lean, back fat, prime cuts yield), transportation of time from hatchery to farm (for chickens, turkeys, etc.), transportation time from farm to a processing plant, age of mother at time of birth (or egg laying), and difference of weight between biological mother and father.

According to one embodiment, the method further includes the step of performing cluster analysis of historical data. According to one embodiment, the historical data includes stored data related to the variables provided herein. The historical data may be obtained from smart farms all over the world. According to one embodiment, the cluster analysis is accomplished through the use of machine learning or application of artificial intelligence. The result of the cluster analysis is a recommendation of a selection of smart farms that represent variability within an entire system of smart farms across the world. Sensors are then assigned to one or more (or all) of the recommended selection of smart farms.

According to one embodiment, real-time livestock data is gathered from the sensors at one or more of the selected smart farms that represent variability within an entire system of smart farms. Such smart farms may be located in various, specific locations around the world. Such real-time livestock data may be provided in real-time via one or more sensors, cameras, or other devices on a smart farm or stored and provided at a later time. According to one embodiment, the real-time livestock data is obtained from a plurality of sensors located on or around a plurality of livestock animals at the smart farm. The one or more smart farms may include one or more barns or houses equipped with one or more sensors and devices to measure the performance of the livestock animals. According to one embodiment, the plurality of sensors transmits a particular code associated with an individual livestock animal.

According to one embodiment, the method further includes the step of analyzing the real-time livestock data from sensors at one or more select smart farms. According to one embodiment, one or more statistical models may be utilized to analyze the data as provided herein. According to one embodiment, the statistical model is one or more of lineal regression, logistical regression, logarithmical regression, survival analysis, analysis of variance (ANOVAS), principal component analysis, autoregressive integrated moving average (ARIMA), machine learning predictive model, or any combination thereof. According to one embodiment, the machine learning predictive model is one or more of decision trees, regression trees, random forest, gradient boosting machine, support vector machine, neural network, Bayesian network, or any combination thereof.

According to one embodiment, the method further includes the step of building or training an artificial intelligence supervised learning model. According to one embodiment, the artificial intelligence supervised learning model seeks to predict and otherwise explain any variability in performance based on the collected real-time data. These learning models include any model provided herein as well as ensemble learning, deep learning, time series modeling, or Bayesian statistics. The real-time data may be extracted, transformed, loaded and integrated onto a database such as, for example, a relational database.

According to one embodiment, the capacity of the leaning model may be tested on training, development and testing datasets. The training model may be used to predict and otherwise explain the percentage of the variance in variables. According to one embodiment, changes to variables (e.g., treatments) may be assigned to smart farms identified by the cluster analysis. According to one embodiment, the evolution of trials may be tracked using the real-time data collected on a daily basis, such as feed intake. The results from the change in variables may be adjusted to take into account the variance explained by the learning model. The change in variables may, in turn, produce real-time data that is analyzed using classical statistical analysis to reach conclusions about the effect of the treatments.

According to one embodiment, the method further includes the step of generating a customized research report that includes the results of the analysis. According to one embodiment, the report includes a suggested modification to one or more of the variables provided herein such as feeding method, feeding schedule, environmental temperature, water pH, water temperature, water quality, ammonia levels in environment, air quality, barn flooring quality, feeder type, feed quality, feed size, nutritional levels and requirements, medication, medication delivery method, and distance of livestock from other barns and/or packing plants. According to one embodiment, the report may include one or more suggestions for the livestock owner regarding the health and well-being of the livestock, including suggestions for improvement. According to one embodiment, the report provides an adaptive and customized nutritional or/and health plan. The report may provide suggestions as to changes in the diet/medication for a particular livestock animal or a group of livestock animals. The changes to diet include, but are not limited to, changes in protein intake and overall caloric intake. According to one embodiment, the report provides various suggestions for the livestock producer regarding medical disease diagnosis and treatment regimen.

According to one embodiment, the method further includes the steps of transmitting the research report wirelessly to the livestock producer; and confirming or denying acceptance of the research report by the livestock producer. According to one embodiment, upon acceptance of the report, the method further includes the step of implementing the suggested modification to one or more variables by the livestock producer. According to one embodiment, upon completion of implementation of modifications, the method includes the step of uploading or otherwise transmitting data related to the outcome of implementation of changes to one or more variables to a cloud configuration or central database. According to one embodiment, the data related to the outcome of implementation of changes to one or more variables is made available for use as historical data in a virtual research model.

### Digital Research System

According to one aspect, a system for digital research is provided. The digital research system includes at least one server. The at least one server may be located in a cloud configuration. According to one embodiment, the digital research system includes at least one database. According to one embodiment, the system includes at least one computer coupled to the at least one server and database. The at least one computer may be located in a cloud configuration and coupled or in communication with the at least one server and database. The computer includes at least one processor and memory for analyzing the data as provided herein.

According to one embodiment, the system for digital research further includes a plurality of smart farms for historical data production. The smart farms for historical data production may be subject to cluster analysis as provided herein. The historical data may be stored on one or more central or remote servers for access when cluster analysis is performed. The server for historical data may be stored on one or more servers in a cloud configuration.

According to one embodiment, the system for digital research further includes at least one livestock producer interface at a smart farm including a data entry system. The livestock producer interface may be in wireless communication with a gateway and the at least one server. According to one embodiment, the livestock producer interface allows the livestock owner to enter individual livestock input data regarding one or more of the data variables provided herein. According to one embodiment, at least one server, memory, artificial intelligence component, and processor are configured to extract, transform, load and otherwise process one or more of producer input data, sensor data, historical data from the plurality of smart farms, and any normalized data.

The system further includes at least one livestock sensor at the smart farm. The livestock sensor may be coupled to a gateway that is in wireless communication with the at least one server. According to one embodiment, the one or more sensors may be coupled to or wirelessly connected to at least one custom electronic board. According to one embodiment, the electronic board filters a data signal from the sensor and transmits the data signal to the gateway. The gateway, in turn, transmits the data signal wirelessly (e.g., via internet or through cellular network) to the at least one server, database processor and memory. According to one embodiment, the livestock sensor data may be viewed via the livestock owner interface in real-time. According to one embodiment, the at least one livestock sensor at a smart farm wirelessly transmits sensor data in real-time including data related to at least one of the variables provided herein as well as data related to one or more of livestock activity level, livestock ammonia level, body temperature, body weight, water intake, body pH, water pH, water quality, carbon dioxide levels in the environment or in the livestock body, water consumption, environmental temperature, environmental humidity, rain quantity, wind speed, and trough water temperature.

According to one embodiment, one or more sensors are located throughout the smart farm where livestock are located. The one or more sensors may be installed in various locations throughout the livestock's environment. According to one embodiment, the one or more sensors are installed inside or outside a barn or stable. According to one embodiment, the one or more sensors are installed outside a barn or stable such as, for example, in a pasture or grazing area where the livestock reside during daytime hours. According to one embodiment, the at least one sensor detects data pertaining to one or more of the variables provided herein as well as one or more of livestock activity level, livestock ammonia level, body temperature, body weight, water intake, body pH, water pH, water quality, carbon dioxide levels in the environment or in the livestock body, water consumption, environmental temperature, environmental humidity, rain quantity, wind speed, and trough water temperature. According to one embodiment, certain sensor data is obtained through various commercial sensors. According to one embodiment, a sensor containing a statistical-based algorithm is utilized to measure livestock physical activity.

The system further includes an artificial intelligence component or machine learning component configured to analyze one or more variables and predict an effect of the selected variable. The system further includes a memory and processor in wireless communication with the server, livestock producer interface, at least one livestock sensor and artificial intelligence component.

According to one embodiment, the system further includes at least one database coupled to or in wireless communication with a gateway that is in wireless communication with the at least one server, memory, artificial intelligence component, and processor. According to one embodiment, a database is provided that stores and maintains various standardized data with respect to each of the health profiles of particular livestock types and the genetic information of particular livestock types (herd or individual livestock). According to one embodiment, the database maintains one or more licenses to access one or more data services which aid in and provide data related to disease diagnosis, prescriptions medicines, health assessment data and genetic data. According to one embodiment, the aforementioned information is received, standardized or normalized and stored for later use. According to one embodiment, all standard data is cleaned and aggregated before further processing.

According to one embodiment, the data and reports as provided herein is entered into or received into a cloud pipeline system. According to one embodiment, the cloud pipeline system includes at least one database, server, processor and memory to receive, analyze, process, and store the various data as provided herein. According to one embodiment, the cloud pipeline system includes more than one or a plurality (e.g., cluster) of databases to receive, process and store the various data as provided herein. According to one embodiment, the cloud pipeline system includes more than one or a plurality (e.g., cluster) of servers to receive, process and store the various data as provided herein. According to one embodiment, the cloud pipeline system includes at least one computer to receive, analyze, process, detect anomalies, and store the various data as provided herein. According to one embodiment, at least one server is configured to receive, process and store the various data as provided herein. According to one embodiment, at least one database is configured to receive, process and store the various data as provided herein. The number of databases, servers, computers or a combination thereof is scalable and varies depending on data size. According to one embodiment, data, such as real-time data, may be extracted, transformed, loaded and integrated on a database such as a relational database.

According to one embodiment, a report is generated and provided by the methods and systems provided herein. The report may include various identifying information such as a client or user identification number and time stamp. According to one embodiment, the report is provided to the livestock producer wirelessly via a livestock producer interface. According to one embodiment, the report is provided wirelessly via email or stored on a central database for central access.

According to one embodiment, data and reports, such as sensor data as provided herein, may be managed and stored in a NoSQL (“non SQL” or “non-relational”) database such as that provided by Apache Cassandra. According to one embodiment, the NoSQL database provides a mechanism for storage and retrieval of data and reports as provided herein that is modeled in means other than the tabular relations used in relational databases.

According to one embodiment, the virtual and digital methods and systems provided herein may be combined to work together to improve livestock health outcomes and performance. According to one embodiment, the virtual and digital methods provided herein may be run concurrently to work together to improve livestock health outcomes and performance. According to one embodiment, one or more steps from the virtual and digital methods provided herein may be combined to work together to improve livestock health outcomes and performance.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be apparent to those skilled in the art, and all modifications that do not depart from the spirit of the disclosure are intended to be included with the scope of the appended claims.

### Example

### Promotion of Broiler Performance—Digital Research Method

The digital research method provided herein was utilized to promote broiler performance through the reduction of gut inflammation.

The digital method was performed by initially forming a hypothesis regarding a variable to change. The hypothesized variable included enzymes in combination with a mixture of essential oils and organic acids. Next, broiler data was gathered from a broiler operation with broiler farms all across the country of Colombia. A cluster analysis was performed using performance key process indicators such as mortality rate, feed to gain ratio, average daily weight wain, final weight and flock size. Three groups of smart farms were identified for real-time livestock data collection. The first smart farm group was characterized for capabilities to produce heavy weight broilers with good performance. The second smart farm group was able to achieve similar performance for lightweight broilers. Finally, the third smart farm group produced broilers from different weight profiles with deficient performance measured across several indicators. A cluster balanced sample of smart farms was selected. If not already present, sensors were installed in sampled smart farms. Sensors installed were configured to measure air temperature, relative humidity, water consumption, water pH, water temperature, wind speed, light intensity and the broiler average weight on a daily basis. Real-time broiler data from several other sources (silos) was received. This real-time data included embryo diagnosis from the hatchery, feed composition, farm specifications, serologies, necropsies and performance from the broiler parents. Data from all of the silos was extracted, transformed, loaded an integrated on a relational database. Data from the smart farm sensors was also integrated into the database. A machine learning predictive model was trained to predict the key variables (e.g., performance indicators) using all the information available on the integrated database. The capacity of the machine learning predictive model was tested on training, development and testing datasets. The machine learning predictive model was able to explain a grade the percentage of the variance in the key variables related animal performance.

A customized research report was produced that included the results of the analysis of the real-time livestock data including recommendations to improve livestock health outcome and performance. The report assigned treatments or otherwise changes to the variables for the smart farms with sensors balanced across the three groups. The evolution of the trials was tracked using the sensors and information collected on a daily basis, such as feed intake. The performance results from the treatments were adjusted to take into account the variance explained by the machine learning predictive model. The adjusted indicators were analyzed using classical statistical analysis to conclude about the effect of the change in variables (e.g., treatments). The variable change was found to significantly reduce the feed to gain ratio, while it had no effect on the average daily weight gain nor the mortality rate. A final report on the effect of the change in variable was then transmitted to the broiler operation owner.

We claim:

1. A method of conducting digital research to improve livestock health outcomes, the method comprising
forming a hypothesis regarding what changes may be made to one or more variables related to livestock nutrition, environment, health status, or animal performance to improve livestock health outcomes for a particular livestock producer;
performing a cluster analysis of historical data related to the one or more variables to determine one or more smart farms for real-time livestock data collection;
obtaining real-time livestock data from the selected one or more smart farms;
analyzing the real-time livestock data to test the hypothesis; and
generating a customized research report that includes results of the analysis of the real-time livestock data including one or more recommendations to improve livestock health outcomes.

2. The method of claim 1, wherein the real-time livestock data is obtained from a plurality of sensors located on or around a plurality of livestock at the smart farm.

3. The method of claim 2, wherein the plurality of sensors transmits a particular code associated with an individual livestock animal or individual smart farm.

4. The method of claim 1, wherein the step of analyzing the real-time livestock data is accomplished by one or more statistical models.

5. The method of claim 1, wherein the cluster analysis utilizes historic data obtained from a plurality of smart farms located throughout one or more global regions.

6. The method of claim 1, further comprising the steps of:
transmitting the research report wirelessly to the one or more smart farms; and
confirming or denying acceptance of the research report by the one or more smart farms.

7. A method for conducting virtual research to predict the outcome of a selected variable of interest and improve livestock health outcomes, the method comprising the steps of:
(a) obtaining livestock data from a plurality of livestock producers, the livestock data related to one or more variables pertaining to livestock nutrition, livestock environment, livestock health or livestock performance;
(b) transmitting the livestock data to a cloud computing environment;
(c) analyzing the livestock data in the cloud computing environment with one or more analysis models; and
(d) generating a customized report that includes the results of the analysis, the results including an estimate or prediction of an expected improvement in livestock health outcome by changing one or more variables.

8. The method of claim 7, wherein the selected variable of interest is selected from the group consisting of microbiome, feeding method, feeding schedule, medical history, breed, breeding status, age, body condition, genetics, gender, environmental temperature, humidity, water pH, water temperature, water quality, ammonia levels in environment, air quality, barn flooring quality/type, feeder type, feed quality, feed size, nutritional levels and requirements, disease, medication, medication delivery method, distance of livestock from other barns and/or packing plants, growth performance, and carcass characteristics.

9. The method of claim 7, wherein the livestock data comprises data obtained from the Internet of Animals.

10. The method of claim 7, wherein the step of analyzing the livestock data includes applying artificial intelligence to the livestock data in a plurality of data processing modules within a cloud computing environment in which the plurality of data processing modules are executed in conjunction with at least one processor, the data processing modules and artificial intelligence configured to continuously evaluate data from a plurality of livestock producers to estimate or predict an expected improvement in livestock health outcome by changing one or more variables.

11. The method of claim 7, further comprising the step of:
transmitting the report to an end user, wherein the end user may accept or deny the report.

12. The method of claim 11, wherein upon acceptance of the report, the method comprises the steps of:
implementing the change to one or more variables in a field trial;
analyzing the data produced from the field trial; and
retraining the one or more of the analysis models.

13. The method of claim 11, wherein upon denial of the report, the method comprises the step of:
repeating steps (a)-(d) until one or more variables dependent on improvement through several simulations is fulfilled or until a maximum number of iterations have been raised.

14. A system for digital research comprising:
at least one database;
at least one server;
at least one livestock producer interface including a data entry system, the livestock producer interface in wireless communication with a gateway and the at least one server;
at least one livestock sensor, the livestock sensor coupled to a gateway that is in wireless communication with the at least one server and the at least one database;
an artificial intelligence component configured to analyze one or more variables and recommend a change to one or more variables; and
a memory and processor in wireless communication with the database, server, livestock producer interface, at least one livestock sensor and artificial intelligence component.

15. The system of claim 14, further comprising at least one livestock scale or camera, the livestock scale or camera coupled to a gateway that is in wireless communication with the at least one server, memory and processor.

16. The system of claim 14, wherein the at least one server, memory, artificial intelligence component, and processor are configured to process one or more of producer input data, sensor data, and any normalized data to recommend a change to one or more variables.

17. A system for virtual research comprising:
a cloud computing environment comprising a statistical model component configured to analyze stored data and estimate or predict an expected improvement in health outcome by changing one or more variables; and
one or more databases comprising stored data, the one or more databases in wireless communication with the cloud computing environment.

18. The system of claim 17, wherein the one or more databases includes access to the Internet of Animals.

19. The system of claim 17, wherein the statistical model component comprises artificial intelligence.

20. The system of claim 17, further comprising a dashboard, the dashboard in wireless communication with the cloud computing environment.
